(19) United States
(12) Patent Application Publication
(10) Pub. No.: US 2010/0222729 A1
(43) Pub. Date: Sep. 2, 2010

(54) CERVICAL TRACTION TREATMENT

(76) Inventors: Lily Chin, Las Vegas, NV (US); Thomas Wong, Las Vegas, NV (US)

Correspondence Address: GREENBERG TRAURIG (LV), 3773 HOWARD HUGHES PARKWAY, Suite 400 North, LAS VEGAS, NV, 89169, US

(21) Appl. No.: 12/396,143

(22) Filed: Mar. 2, 2009

(51) Int. Cl. A61F 5/00

(52) U.S. Cl. 602/32; 602/36

(57) ABSTRACT

Described is a method of providing headache and migraine relief by applying traction to the cervical spine of a human subject. The human subject may be placed in a supine or prone position with an attachment means positioned around the skull region of the human subject. The attachment means is coupled to a traction means, which provides traction forces for stretching the cervical spine and providing relief from headache and migraine pain associated with cervical compression. Various treatment regimes are possible depending on the patient size and symptoms.

### FIELD OF THE INVENTION

The embodiments of the present invention relate to physiotherapy treatment, more specifically, to methods of applying traction forces to the cervical spine of a human subject.

### BACKGROUND

It is widely theorized and acknowledged that certain types of headaches and migraines originate intra-cranially. It is far less understood that some migraine headaches and other cranial maladies have their origins in the cervical spine. Specifically, migraines may result from compression of the cervical spine structures such as the intervertebral discs. The operant postulate is that migraine headaches and other illnesses of the head region can result from an inflammation of cervical structures (including the C1 through C7 vertebrae and perhaps even those of the upper thoracic spine as well as neck muscles, connective and other soft tissues). Conventional medical treatment of headaches and migraines have often focused on pharmacological solutions with an emphasis on intracranial causes, but have generally overlooked the neck and cervical spine elements as possible culprits.

As such, there exists a need for methods of providing therapeutic relief from headaches and migraines originating from the cervical spine.

### SUMMARY

Accordingly, a first embodiment of the present invention discloses a method for applying forces to the cervical spine of a human subject, the method comprising: positioning an attachment means around a skull of the human subject; coupling the attachment means to a traction means; and applying traction forces to the cervical spine of the human subject using the traction means. Depending on the embodiment, the attachment means may include an adjustable head harness, head halter, neck wrap, occipitomandibular halter or cervical collar and may be positioned around the mandible and/or occiput of the human subject, while the traction means may include a traction device or machine. Depending on the patient, the traction forces can be gradually or intermittently applied to the cervical spine including the neck region and the C1 through C7 vertebrae and the traction forces being applied to the human subject can be horizontal, vertical, lateral or longitudinal forces.

Other variations, embodiments and features of the present invention will become evident from the following detailed description, drawings and claims.

### BRIEF DESCRIPTION OF THE DRAWINGS

FIG. 1 illustrates a perspective view of a human subject placed in a supine position undergoing a cervical traction treatment according to an embodiment of the presently disclosed invention; and

FIG. 2 illustrates a perspective view of a human subject sitting in an upright position undergoing a cervical traction treatment according to an embodiment of the presently disclosed invention.

### DETAILED DESCRIPTION

It will be appreciated by those of ordinary skill in the art that the invention can be embodied in other specific forms without departing from the spirit or essential character thereof. The presently disclosed embodiments are therefore considered in all respects to be illustrative and not restrictive.

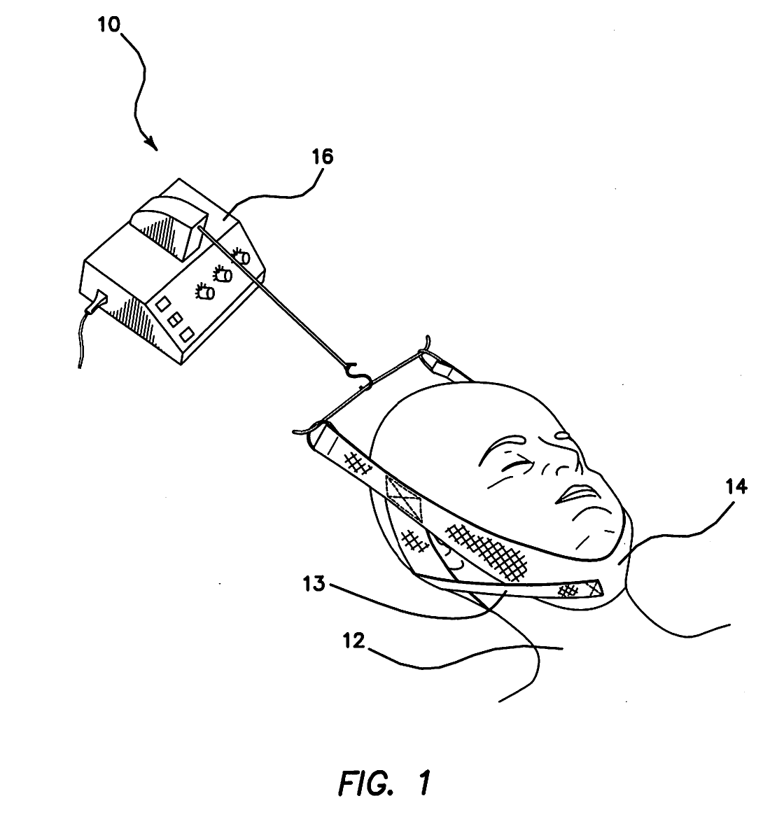

Initial reference is made to FIG. 1 illustrating a perspective view of a human subject 12 placed in a supine position undergoing a cervical traction treatment 10 according to a first embodiment of the presently disclosed invention. As shown in FIG. 1, the human subject 12 is placed in a supine position or lying down on his or her back. In another instance, the human subject 12 may be placed in a prone or downward facing position. An attachment means 14 constructed of cloth, foams, canvas, flannel or other suitable material is positioned around the base of the skull and/or mandible of the human subject 12 without impinging on the throat or impeding air flow.

As shown in FIG. 1, the attachment means 14 covers the occiput or other parts of the head or skull including the mandible or jaw of a human subject. In one embodiment, the attachment means 14 includes a head halter or an adjustable head harness utilizing any necessary straps, buckles, loops and belts. In other embodiments, the attachment means 14 includes a neck wrap, sling or cervical collar having stabilizing straps and foams. Although the attachment means 14 as shown in FIG. 1 covers both the occiput and the mandible, it is understood that the attachment means 14 may cover only the mandible or only the occiput, such as by means of an occipital or mandible halter. It will be appreciated by one skilled in the art that any suitable attachment means 14 positionable around the skull, jaw and/or neck of the human subject 12 may be employed.

While one end of the attachment means 14 is positioned around the head and/or neck of the human subject 12, the opposite end of the attachment means 14 is coupled to a traction means 16. Specifically, the traction means 16 includes a traction machine or device 16 capable of applying traction forces. In one embodiment, the traction device 16 is capable of providing intermittent or gradual traction forces. The traction device 16 may also incorporate timers and other suitable programming devices with the ability to alternate, fluctuate, increase or decrease the amount of traction forces to be applied. Such programming may be facilitated by software and/or hardware components commonly known in the art. The software and/or hardware components can be integrated within the traction device 16 or externally implemented.

Once the attachment means 14 couples the human subject 12 and the traction device 16, intermittent or gradual traction forces may be applied to the neck thereby stretching the cervical spine structures of the human subject 12. In general, the cervical spine structures include the neck region, any relevant bones and the C1 through C7 vertebrae. In one embodiment, sufficient traction force is applied by the traction machine 16 to decompress the cervical spine anatomy and, theoretically, provide rapid and significant relief to the cervical spine structures. This therapeutic treatment may accordingly relieve any associated headaches or migraines. In one embodiment, the traction force is greater than 35 pounds per square inch for an average adult subject and the treatment can last 15-20 or more minutes per session with no limit on the frequency of treatment. The traction force may be adjusted based on the body weight of the human subject 12 such that treatments can be carried out on adults and children of all ages. Likewise, treatment time can be varied as well as the intensity and frequency of treatment based on a provider's subjective feelings of tolerance and comfort. Cervical traction can vary in regards to frequency, intensity and length of time depending on patient acceptance and need. To demonstrate the benign nature of cervical traction, it is possible that some patients 12 may fall asleep during the procedure. The upper limit of traction force is that at which the patient's body 12 begins to move toward the traction device 16.

In other instances, the body of the subject 12 may begin to move toward the direction of the traction machine 16 because of the traction force being applied. In other words, the subject 12 may be pulled toward the traction device 16 because of the tugging force exerted by the machine 16 on the subject 12. Straps or other securing means (not shown) may be utilized to fasten the subject 12 in ensuring that the subject 12 does not move about while undergoing treatment.

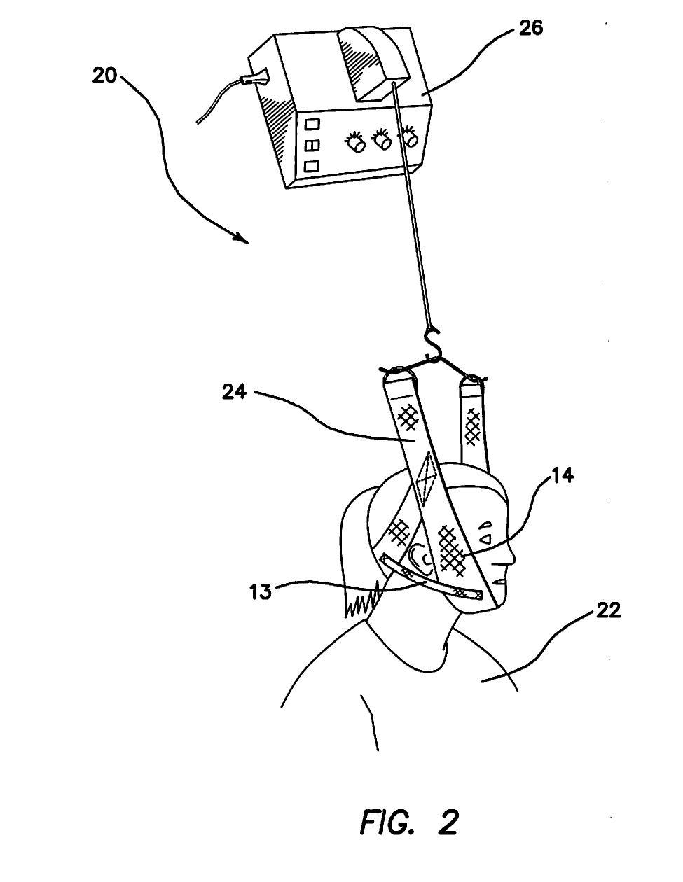

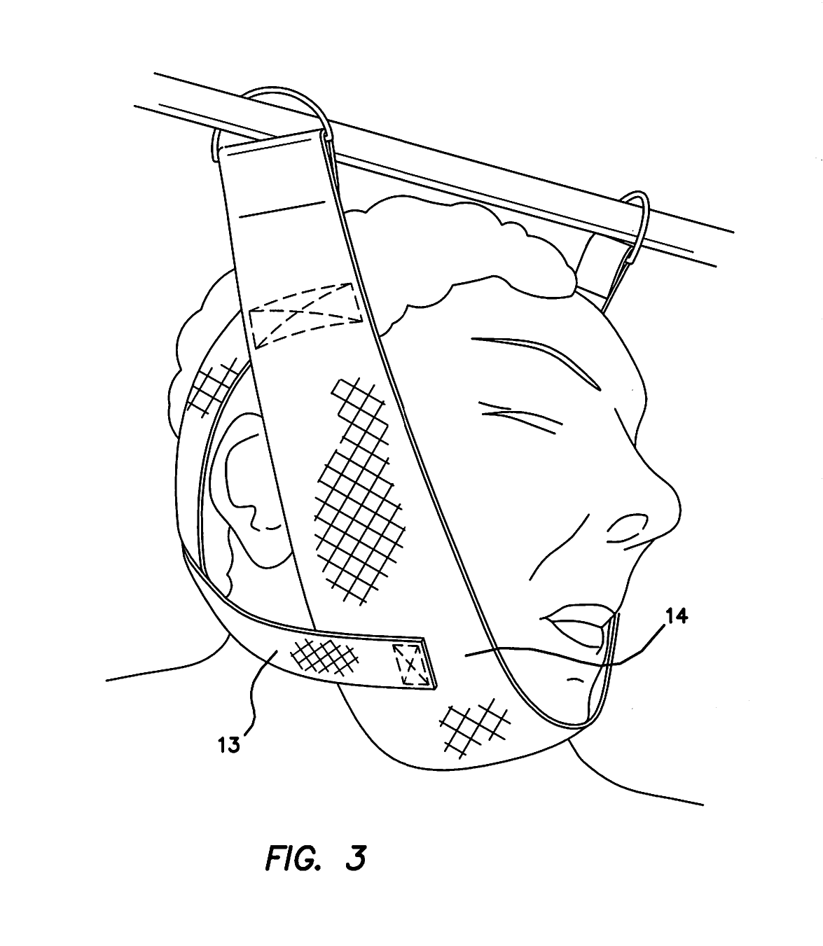

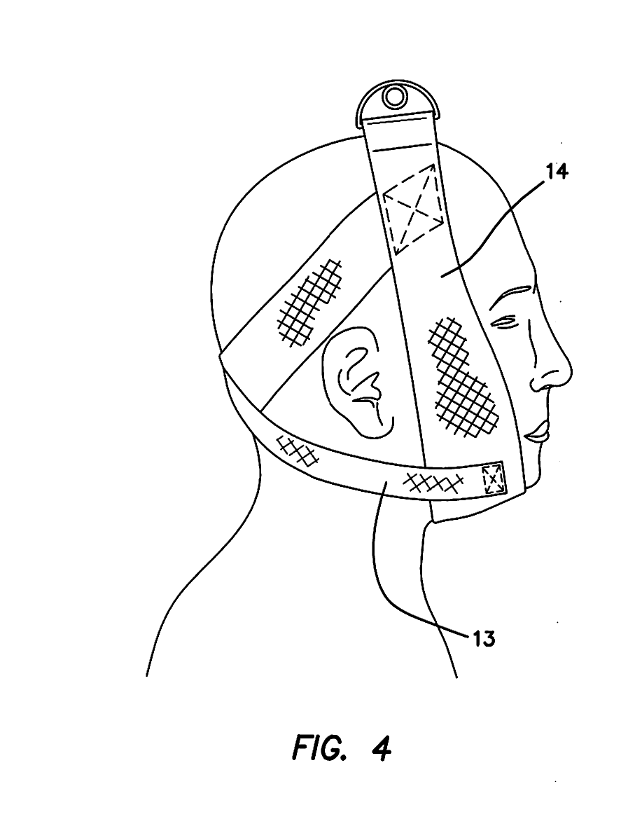

Reference is now made to FIG. 2 illustrating a perspective view of a human subject 22 sitting in an upright position undergoing a cervical traction treatment 20 according to a second embodiment of the presently disclosed invention. Like the previous embodiment where longitudinal traction forces are applied to the human subject 12 by the traction device 16, this presently disclosed embodiment includes a human subject 22 sitting in an upright position coupled with an adjustable head harness 24, which is subsequently coupled to a traction machine 26. The vertical traction forces being applied by the traction machine 26 in this embodiment can also help to provide relief to the cervical spine of the human subject 22 by stretching the neck region and the C1 through C7 vertebrae. The treatment regime in this instance can be similar to those described above wherein the traction force, duration, intensity and frequency can be adjusted accordingly.

It will be appreciated by one skilled in the art that the proposed embodiments are, in general, relatively simple, flexible and safe if basic precautions are followed. Contraindications include previous cervical spinal surgery; history of cervical vertebral fracture; dislocation and subluxation; cervical spine tumor; a history of possible cervical vertebral instability or the possibility of producing such instability. As almost all medical therapies can cause complications or untoward effects, care must always be taken to monitor closely all patients undergoing cervical traction. Likely culprits as a result of cervical traction are the production of temporo-mandibular symptoms or disease; or the aggravation of a previously existing temporo-mandibular condition. Similarly, production or aggravation of dental maladies is also possible. Otherwise, this treatment is relatively free of complication.

Described below are three case studies of patients previously subjected to the embodiments disclosed in the present invention.

Case study 1 involves a 36-year old Caucasian female with a well-established history of classic migraine headaches dating from her mid-twenties. Her migraines were usually unilateral, most often right-sided; lasting more than a day, usually several. Limited relief was best obtained with bed rest. She frequently experienced concurrent nausea and vomiting. She took anti-inflammatory medications for relief, and occasionally narcotics. On at least one extraordinarily severe episode, she visited the local hospital emergency department for a narcotic injection of Demerol (meperidine).

She presented to the clinic in the throes of a typical severe migraine headache episode experiencing marked associated photophobia and phonophobia. She was given intermittent cervical traction of 35-40 pounds of pressure per square inch. The treatment session lasted approximately 30 minutes. Upon cessation of therapy, she experienced significant relief of pain and all other symptoms. She returned the following day much improved for a repeat treatment. She claims to have never experienced such rapid or thorough relief of her migraine headaches.

Case study 2 involves a 53-year old Caucasian male with a history of classic migraines since mid-adolescence, and increasing in severity and frequency of the past decade. He has seen “countless” physicians, including chiropractors and neurologists, seeking relief. CT and MRI scans of his brain have revealed no evidence of physical disease. His medications include ibuprofen and other non-steroidal anti-inflammatory formulations, and combination medications that included a barbiturate, codeine and acetaminophen or aspirin. He has used several of the newest anti-migraine medications and even occasional anti-epileptic medicines.

On the day of presentation, his treatment regime was intermittent cervical traction of approximately 30 minutes duration at 55 pounds per square inch with excellent relief of symptoms. He received another traction session the following day, even though symptoms had largely disappeared. Repeat treatment of cervical traction once or twice per week in the ensuing month have eliminated his migraines entirely.

Case study 3 involves a 72-year old Asian female with right-sided migraine headaches whose onset occurred after a fall she sustained over thirty years ago. Her headaches, while severe themselves, were also regularly associated with significant vertigo, nausea, and vomiting that immobilized her for days. Neurological exams and brain scans have been negative. Anti-inflammatory medications, anti-depressants and various other headache medications in the past have provided little notable relief. Except for treated hypertension, her health was otherwise unremarkable. Examination of her neck revealed no obvious pathology.

She presented to the clinic in the midst of a debilitating headache and vertigo of several days duration. Intermittent cervical traction for 30 minutes at 30 pounds per square inch produced immediate substantial relief as has never been previously experienced through any other method of therapy. She has ceased all medications as well as other treatment modalities including visits to various doctors for prescription drugs, or consultations. Her following treatment was a maintenance regimen of periodic cervical traction as warranted by symptoms.

In the above three case studies, intermittent cervical traction was applied gradually to the cervical spine. The force of cervical traction was dependent upon patient size and individual tolerance (in some instances, forces considerably greater than 60 pounds per square inch are acceptable). Prior to any neck traction, a detailed history has been taken to preclude any past or present cervical spinal structural disease such as tumor, dislocation, fracture and the like. Post-treatment, all patients were prescribed a cervical collar and bed rest to further minimize neck movement and inflammation.

Although the invention has been described in detail with reference to several embodiments, additional variations and modifications exist within the scope and spirit of the invention as described and defined in the following claims.

What is claimed is:

1. A method for applying forces to the cervical spine of a human subject, the method comprising:
positioning an attachment means around a skull of the human subject;
coupling the attachment means to a traction means; and
applying traction forces to the cervical spine of the human subject using the traction means.

2. The method of claim 1, further comprising providing attachment means comprising an adjustable head harness, head halter, neck wrap, occipitomandibular halter or cervical collar.

3. The method of claim 1, wherein the traction means includes a traction device or machine.

4. The method of claim 1, further comprising gradually applying the traction forces to the cervical spine of the human subject.

5. The method of claim 1, further comprising intermittently applying the traction forces to the cervical spine of the human subject.

6. The method of claim 1, wherein the cervical spine includes the neck region and the C1 through C7 vertebrae.

7. The method of claim 1, further comprising positioning the attachment means around the mandible and/or occiput of the human subject.

8. A method for applying forces to the cervical spine of a human subject, the method comprising:
positioning an attachment means around the mandible of the human subject;
coupling the attachment means to a traction means; and
applying traction forces to the cervical spine of the human subject using the traction means in combination with the attachment means.

9. The method of claim 8, further comprising positioning the attachment means comprising an adjustable head harness, head halter, neck wrap, occipitomandibular halter or cervical collar.

10. The method of claim 8, wherein the traction means includes a traction device or machine.

11. The method of claim 8, further comprising gradually applying the traction forces to the cervical spine of the human subject.

12. The method of claim 8, further comprising intermittently applying the traction forces to the cervical spine of the human subject.

13. The method of claim 8, wherein the cervical spine includes the neck region and the C1 through C7 vertebrae.

14. The method of claim 8, further comprising positioning the attachment means around the occiput of the human subject.

15. A method for applying horizontal, longitudinal, vertical or lateral forces to the cervical spine of a human subject, the method comprising:
positioning one end of the attachment means around the mandible and/or occiput of the human subject;
coupling a second end of the attachment means to a traction device; and
applying traction forces to the cervical spine of the human subject using the traction device in combination with the attachment means.

16. The method of claim 15, further comprising positioning the attachment means comprising an adjustable head harness, head halter, neck wrap, occipitomandibular halter or cervical collar.

17. The method of claim 15, further comprising gradually applying the traction forces to the cervical spine of the human subject.

18. The method of claim 15, further comprising intermittently applying the traction forces to the cervical spine of the human subject.

19. The method of claim 15, wherein the cervical spine includes the neck region and the C1 through C7 vertebrae.
